(19) United States
(12) Patent Application Publication
(10) Pub. No.: US 2013/0012785 A1
(43) Pub. Date: Jan. 10, 2013

(54) METHOD AND APPARATUS FOR ENABLING CONTINUOUS DATA ACQUISITION ACROSS MULTIPLE STAGES OF CARE

(76) Inventors: Daniel J. LOMBARDI, Verona, WI (US); William S. King, Poynette, WI (US); Ronald Schluter, New Glarus, WI (US); William E. Kolasa, Belleville, WI (US); Patricia Klossner, New Glarus, WI (US); Linda Quinlivan, Lewisville, NC (US); Eric T. Garz, Lodi, WI (US)

(21) Appl. No.: 13/178,275

(22) Filed: Jul. 7, 2011

(51) Int. Cl. A61B 5/00; G06F 15/00

(52) U.S. Cl. 600/301; 702/188

(57) ABSTRACT

A data acquisition device for providing continuous data acquisition across a plurality of stages of care through a plurality of functioning modes is disclosed. The data acquisition device includes two inputs coupled to an instrumentation amplifier, wherein each input provides an input signal to the instrumentation amplifier, a mode selection module coupled to the instrumentation amplifier, operable to select either an AC mode or one of a plurality of DC modes for processing the input signals, wherein the mode is determined based at least in part on analyzing at least one of a plurality of parameters associated with a plurality of stages of care, and a data collection module operable to process the received signals using the selected mode.

### FIELD OF THE INVENTION

Aspects of the present invention relate to data acquisition devices and methods for providing continuous data acquisition across a plurality of stages of care through a plurality of functioning modes.

### BACKGROUND

Currently no portable device is available in the related art that may acquire, store and transmit EEG and other physiological data, throughout multiple stages of care. For example, different stages of care may have different parameter constraints. A device used for long term monitoring may be small, light weight and have a long battery life. By contrast, a device used to monitor a patient during surgery may use a comparatively large amount of power and record data at a comparatively high rate.

In addition, when the technical specifications for recording clinical EEG were established, the goal was to record then known signals from the brain. Due to technical constraints, high and low pass filters were introduced limiting the bandwidth to 0.05 Hz-100 Hz. Unfortunately, these filters eliminated such information as high gamma frequency oscillations, ripple waves and DC shifting signals that may provide useful cortical information for identifying certain epileptic, stroke, traumatic brain injury pathophysiology.

Thus, there is a need in the art for a single data acquisition device that is capable of providing continuous data acquisition across a plurality of stages of care, through a plurality of functioning modes. Additionally, there is also a need for a data acquisition device that has the expanded capability to record this information.

### SUMMARY

The following presents a simplified summary of one or more aspects of the present invention in order to provide a basic understanding of such aspects. This summary is not an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later.

Aspects of the present invention provide a data acquisition device, comprising two inputs coupled to an instrumentation amplifier, wherein each input provides an input signal to the instrumentation amplifier, a mode selection module, coupled to the instrumentation amplifier, operable to select either an alternating current (AC) mode, and/or one of a plurality of direct current (DC) modes for processing the input signals, wherein the mode is determined based at least in part on analyzing at least one of a plurality of parameters associated with a plurality of stages of care, and a data collection module is operable to process the received signals using the selected mode.

Aspects of present invention also provide a method of continuously acquiring data, the method comprising receiving a selection of a first mode of operation from a plurality of modes, wherein the mode is determined based at least in part on analyzing at least one of a plurality of parameters associated with a plurality of stages of care, receiving input signals from two inputs, processing the received input signals using the selected first mode of operation to generate a first data set, receiving a selection of a second mode of operation from the plurality of modes, wherein the second mode is different than the first mode, and processing the received input signals using the selected second mode of operation without interruption in receiving the input signals to generate a second data set.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### DESCRIPTION OF THE DRAWINGS

FIG. 1 is a block diagram of an example data acquisition device that can continuously acquire data over a plurality of stages of acre, according to an aspect;

FIG. 2 is an example instrumental amplifier circuit design for a data acquisition device, according to an aspect;

FIG. 3 is a flowchart of an aspect of a method of continuously acquiring data over multiple stages of care; and

FIG. 4 is a flowchart of operation of a data acquisition device across multiple stages of care.

### DETAILED DESCRIPTION

Various aspects of methods and apparatuses of the present invention are described more fully hereinafter with reference to the accompanying drawings. These methods and apparatuses may, however, be embodied in many different forms and should not be construed as limited to any specific structure or function presented throughout this disclosure. Rather, these aspects are provided so that this disclosure will be thorough and complete, and will fully convey the scope of these methods and apparatus to those skilled in the art. Based on the descriptions and teachings herein, one skilled in the art should appreciate that that the scope of the disclosure is intended to cover any aspect of the methods and apparatus disclosed herein, whether implemented independently of or combined with any other aspect of the disclosure. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure herein may be embodied by one or more elements of a claim.

Generally, a data acquisition device 100 may be operable to provide continuous data acquisition, without interruption, as a patient moves through various stages of care. Further, the data acquisition device may use multiple modes of operation to acquire data, where each mode may be operable to effectively function in each the different stages of care. For example, data acquisition device 100 may be operable to cover the range from acute intervention in an ambulance/emergency setting and throughout the continuity of care. As such, data acquisition system 100 may provide improved patient safety and hospital experience, due among other things, to increased mobility. Still further, data acquisition system 100 may be used to assist medical practitioners in a variety of practices, such as but not limited to in a veterinary practice.

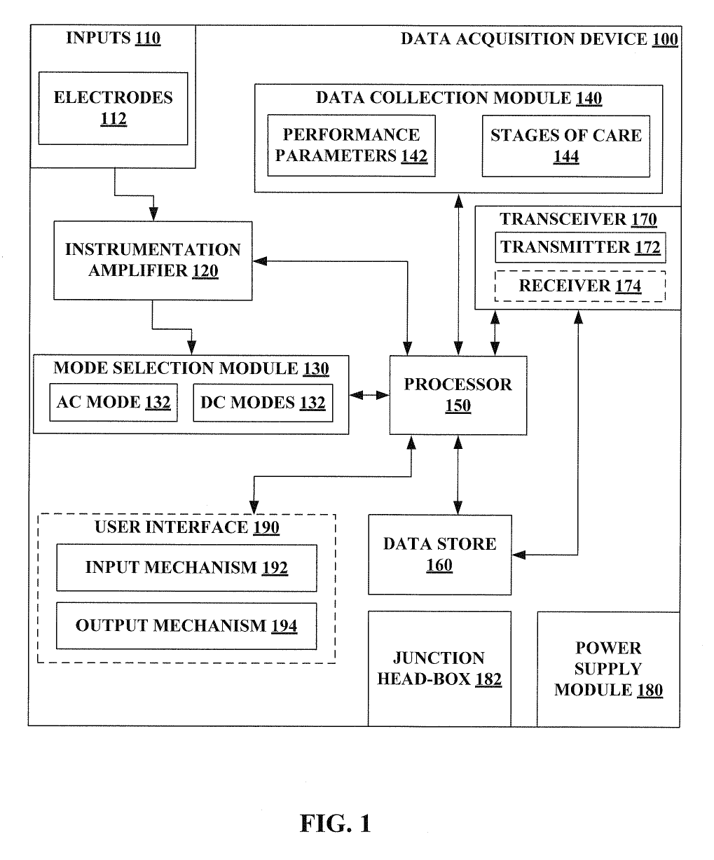

Referring to FIG. 1 a data acquisition device 100 is illustrated. In one aspect, data acquisition device 100 may include inputs 110, instrumentation amplifier 120, mode selection module 130, data collection module 140, processor 150, data store 160, transceiver 170, power supply module 180, junction head-box 182. Additionally, in an optional aspect, data acquisition device 100 may include user interface 190.

In one aspect, inputs 110 may include multiple input feeds operable to provide patient related information. For example, inputs may include electrodes 112 that may be attached to a patient, vital sign monitoring inputs, etc. Input 110 allows for flexible channel counts and amp configurations. In one aspect, inputs 110 may include a channel count range from 32 to 256 to provide connectivity with multiple units and to increase manufacturing efficiency and customer upgradeability. Each channel may have built in impedance measuring capability and light emitting diode (LED) level displays. In one aspect, inputs 110 may include 32 channels with 9 optional bipolar channels for interfacing with various sensors, such as but not limited to, sleep sensors. In another aspect, inputs 110 may include 64 channels for operable for long range epilepsy monitoring. In still another aspect, inputs 110 may be implemented to allow for operability with clinical head-boxes with industry standard 10/10 and/or 19/20 electrode patterns. In another aspect, input 110 may include integrated and removable cortical stimulators. Recording from more channels may improve diagnostic and neurosurgical treatment decision-making.

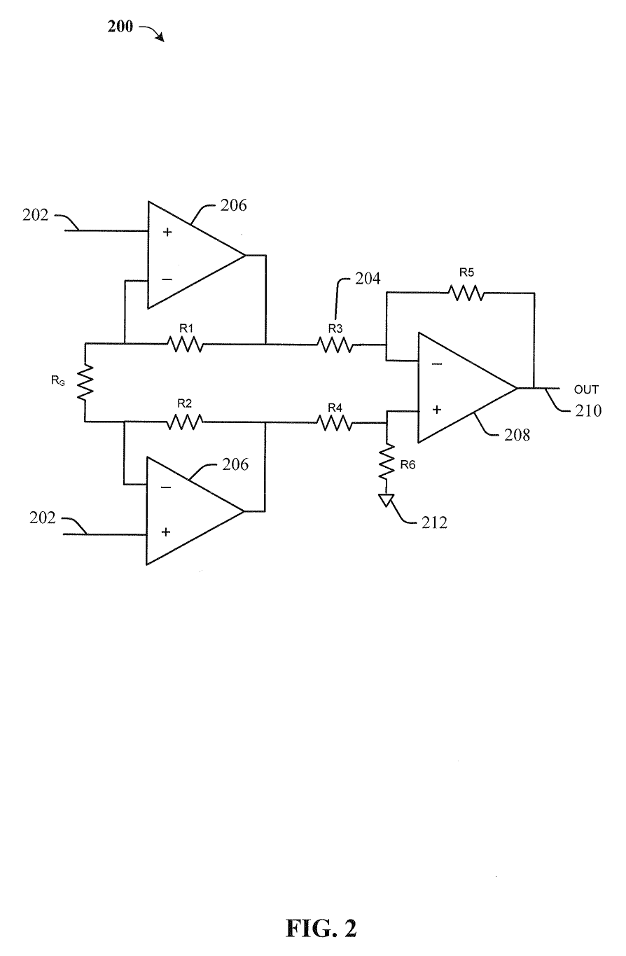

Inputs 110 may be coupled to instrumentation amplifier 120. An example of circuitry for an instrumentation amplifier is provide for reference and discussed is detailed in FIG. 2. In one aspect, instrumentation amplifier 120 may be powered by power supply module 180 using a dual supply.

Mode selection module 130 may include an AC mode 132 and/or a plurality of DC modes 134. In one aspect, AC mode 132 may be selected through use of two AC electrode paths in mode selection module 130. In one aspect, the AC mode 132 may be used for clinical electroencephalography (EEG), long term monitoring (LTM), operating room (OR), intensive care unit (ICU), etc., applications. Further, in one illustrative implementation, AC Mode 132 may be operable to function with a gain of approximately 400 and a sensitivity of about +/−5 mV.

In one aspect, each DC mode 134 may be selected through application of one or more switches in an electrode path in module selection module 130. In another aspect, DC modes 134 may be used for research, sleep study, etc., applications. Further, DC modes 134 may include multiple DC modes of operation, with varying gains and levels of sensitivity. In one illustrative implementation, DC modes 134 may include a first mode with a gain of approximately 1.5 and a sensitivity of about +/−1.3V, a second mode with a gain of approximately 6.0 and a sensitivity of about +/−325 mV, and a third mode with a gain of approximately 12.5 and a sensitivity of about +/−160 mV.

Further, mode selection module 130 may determine a mode of operation based in part of various performance parameters 142, and an applicable stage of care 144. As such, mode selection module 130 may use a single power supply 180 (e.g., 5 VDC) and provide the gains, biasing, and buffering to scale the input signals for an analog to digital converter.

Data collection module 140 may include performance parameters 142 and a plurality on stages of care 144. In one aspect, the data collection module may determine which mode of data acquisition to select based at least in part on analysis performance parameters 142 and stages of care 144. As an example, applicable performance parameters may include: input signal sampling rate, noise in the input signals, a common mode rejection ratio (CMRR) value, cost of equipment, device battery 180 life, device memory 160 capacity, device 100 size, DC offset tolerance, input bias current, bandwidth sensitive range, etc. As another example stages of care 144 may include: clinical electroencephalography (EEG), long term monitoring (LTM), operating room (OR), intensive care unit (ICU), sleep study operation, research study operation, etc. For example, in one aspect, data collection module 140, when combined with a trigger from a stimulator (not shown), may provide evoked potential functionality. Such evoked potential functionality may be used to assist neurosurgeons in locating motor strips before re-secting brain tissue and thereby potentially reducing seizure activity.

For example, recording very low and very high frequency components of the EEG frequency spectrum may be useful in order to efficiently capture physiological data and/or information about a disease state of the brain. As such, data collection module 140 may be operable to support data collection throughout a full band EEG (FbEEG) for recording EEG in both basic science and clinical settings. FbEEG may include bandwidth from zero HZ (e.g., DC) to approximately 6 KHz with sampling rates to approximately 32 KHz and approximately 24 bit resolution. Additionally, for routine clinical EEG, a noise floor of approximately 2 uVpp may be maintained to allow for brain death evaluation. In other words, both AC and DC recordings allow clinicians to evaluate FbEEG recordings while managing power consumption, cost, size, and weight parameters. DC data collection may help predict abnormal seizure events, among other things, so that pre-emptive treatment may be administered. Providing for ultra high EEG frequency data collection may allow for improvements in localizing and diagnosing focal events.

Further, data collection module 140 may be operable to obtain various measures from inputs 110. Such measurements may include, but are not limited to, EEG measurements, TCD measurements, perfusion measurements, ICP measurements, invasive depth EEG measurements, blood pressure measurements, temperature measurements, heart rate measurements, SpO₂ measurements, evoked potential measurements, vital sign monitoring measurements, etc. In another aspect, measurements may be taken to determine a depth of anesthesia.

Processor 150 may comprise processor dedicated to analyzing information received from inputs 110 and/or generating information for transmission by transmitter 172, a processor that controls one or more components of data acquisition device 100, and/or a processor that both analyzes information received from inputs 110, generates information for transmission by transmitter 172, and controls one or more components of data acquisition device 100.

Processor 150 may be operable to receive a selection of a first mode of operation from a plurality of modes, wherein the mode is determined based at least in part on analyzing at least one of a plurality of parameters associated with a plurality of stages of care, and include features for receiving input signals from two inputs, features for processing the received input signals using the selected first mode of operation to generate a first data set, features for receiving a selection of a second mode of operation from the plurality of modes, wherein the second mode is different than the first mode, and features for processing the received input signals using the selected second mode of operation without interruption in receiving the input signals to generate a second data set. Processor 150 may further include at least one processor enabled to perform one or more of the above features.

Data store 160 may be operable to store at least a portion of data collected by data collection module 140. It will be appreciated that data store (e.g., memory 408) described herein may include either volatile memory or nonvolatile memory, or may include both volatile and nonvolatile memory. By way of illustration, and not limitation, nonvolatile memory may include read only memory (ROM), programmable ROM (PROM), electrically programmable ROM (EPROM), electrically erasable PROM (EEPROM), and/or flash memory. Volatile memory may include random access memory (RAM), which acts as external cache memory. By way of illustration and not limitation, RAM is available in many forms, such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM). Data store 160 of the subject devices and methods may include, without being limited to, these and any other suitable types of memory.

Transceiver 170 may include transmitter 172. In one aspect, transmitter 172 may include a wireless transmitter, which may be operable to transmit data, stored in a data store. In such an aspect, transmitter 172 may provide for continuous transmission of data for viewing of trends in data, and for such features as, data mining and distributing of data across a network to limit the number of personnel for a given patient, etc. Additionally in an optional aspect, transceiver 170 may include receiver 174. In such an aspect, receiver 174 may receive commands related to types of data to acquire, store, etc., mode of operation, etc.

Power supply module 180 may include a connection to allow for power to be supplied to device 100 and/or various battery options. For example, device 100 may be powered by multiple battery packs allowing varying battery life for mobile data acquisition. Additionally, or in the alternative, a medical grade power supply 180 may be used, providing patient isolation and producing trickle charging of attached batteries. In one aspect, power supply module 180 may provide sufficient battery life and computer power to allow data acquisition device 100 to operable in a standalone mode. In such an aspect, data collection module 140 may acquire inputs from a variety of bandwidths. For example, data acquisition device 100 may be used in a sports medicine field in which EEG activity and accelerometers outputs may be measured to determine forces involved in various sports related injures, such as head trauma.

Junction head-box 182 may include one or more connections to allow for universal interfacing. In one aspect, junction head-box 182 may provide universal interfaces for electrode caps, connections to legacy head-boxes, connectivity to standard intra-cranial grids, micro-electrode signal conditioning, etc.

Additionally, in one optional aspect, data acquisition device 100 may include user interface 190. User interface 190 may include input mechanisms 192 for generating inputs into data acquisition device 100, and output mechanism 194 for generating information for consumption by the user of the data acquisition device 100. For example, input mechanism 192 may include a mechanism, such as a key or keyboard, a mouse, a touch-screen display, a microphone, etc. Further, for example, output mechanism 194 may include a display, an audio speaker, a haptic feedback mechanism, a Personal Area Network (PAN) transceiver, etc. In the illustrated aspects, the output mechanism 194 may include a display operable to present media content that is in image or video format and/or an audio speaker to present media content that is in an audio format. In one aspect, user interface 190 may video display and/or connectivity to a video display to display outputs associated with data collected by data collection module 140.

As such, data acquisition system 100 provides a single device that is not only operable for all stages of care but that the same device can stay with a patient as they progress through these different stages of care. In other words, data acquisition system 100 provides a portable device with sufficient functionality to travel with a patient as they navigate through their care.

Referring to FIG. 2, an example circuit for a instrumentation amplifier 200 usable in accordance with aspects of the invention is illustrated. In one variation, instrumentation amplifier 200 may be a 3 op-amp instrumentation amplifier. Further, instrumentation amplifier 200 may be designed to interface with patient electrodes (e.g., electrodes 112). Instrumentation amplifier 200 includes multiple resistors 204, two buffer amplifiers 206 and a difference amplifier 208, and receives inputs 202 and generates an output 210. In one aspect, a gain of 10 dB (e.g., 3.16) may be used to set buffer amplifiers. In another aspect, the buffer amplifiers may be selected based on one or more factors, such as, but not limited to, small package, low power, low I-bias, low noise, wide supply rails, unity gain, etc. In one aspect, difference amplifier 208 may be set with a gain of about 0.5 dB (e.g., 1.059). In another aspect, the difference amplifier may be selected based on one or more factors, such as, but not limited to, small size, low power, wide supply rails, high CMRR, etc. Further, instrumentation amplifier 200 may be set to have supply rail values of about +10.5 V DC and −7.5 V DC. Still further, the reference pin 212 may be driven to +2.5 V DC, for example, to allow subsequent components, such as mode selection module 130, to run using a single supply. Thus, driving the in-amp reference pin 212 to 2.5 V DC and allow the next stage to cancel low frequency drift of a bias generating, thereby allowing instrumentation amplifier 200 to act as a DC coupled amplifier.

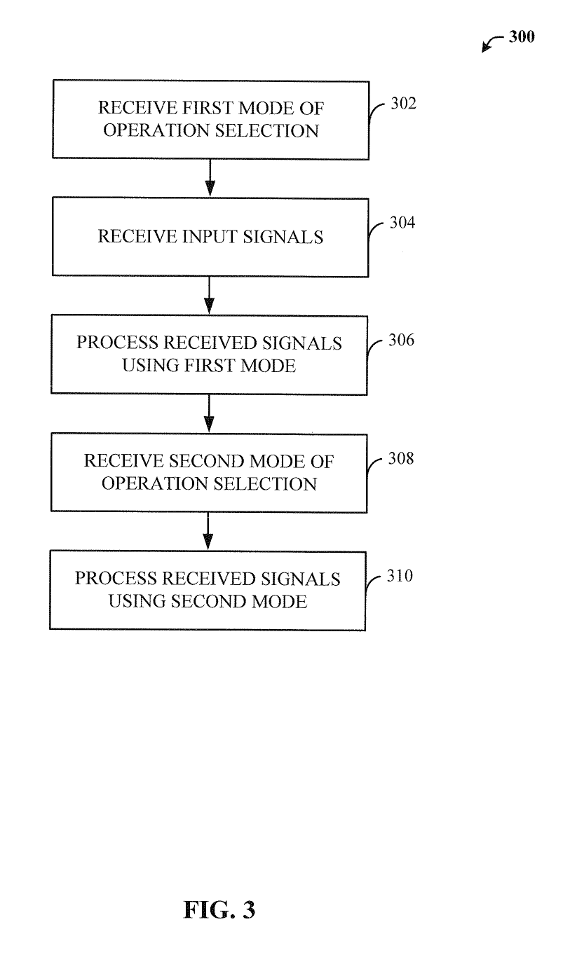

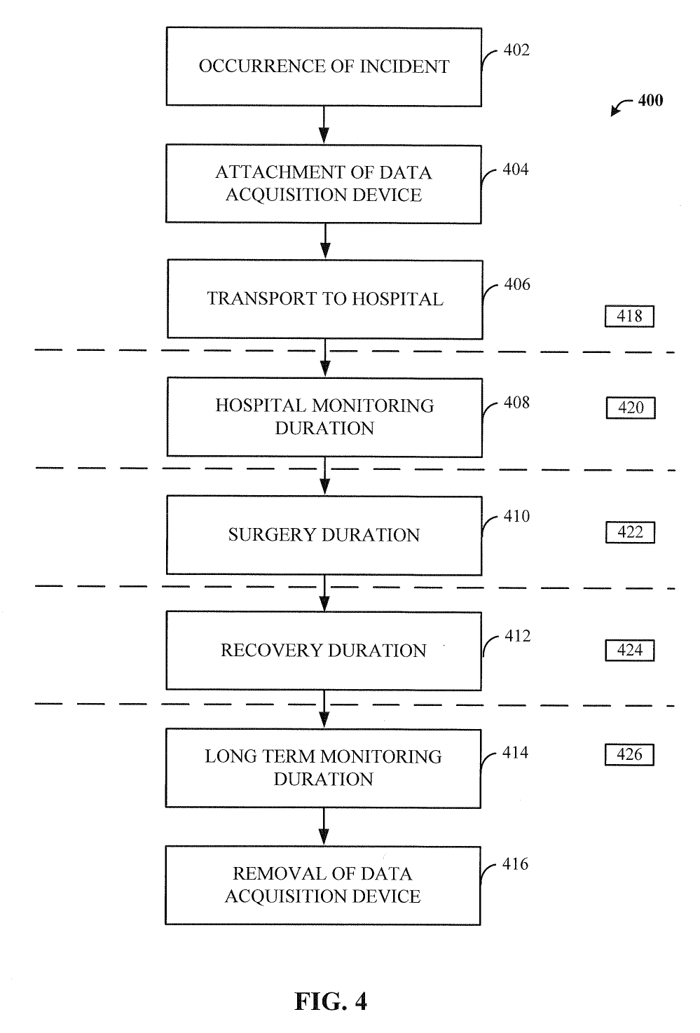

Referring to FIGS. 3 and 4, example flowcharts are shown that define aspects of various methodologies operable in accordance with aspects of the present invention. While, for purposes of simplicity of explanation, the methodologies are shown and described as a series of acts, it is to be understood and appreciated that the claimed subject matter is not limited by the order of acts, as some acts may occur in different orders and/or concurrently with other acts from that shown and described herein. For example, those skilled in the art will understand and appreciate that a methodology could alternatively be represented as a series of interrelated states or events, such as in a state diagram. Moreover, not all illustrated acts may be required to implement a methodology in accordance with the claimed subject matter. Additionally, it should be further appreciated that the methodologies disclosed hereinafter and throughout this specification are capable of being stored on an article of manufacture to facilitate transporting and transferring such methodologies to computers. The term article of manufacture, as used herein, is intended to encompass a computer program accessible from any computer-readable device, carrier, or media.

Referring to FIG. 3, an example flowchart of a method in accordance with aspects of the present invention for enabling continuous data acquisition across multiple stages of care 300 is illustrated. At reference numeral 302, a selection of a first mode of operation is received by a device. The mode selection may be made from a plurality of modes in which the device is operable, for example. In one aspect, the plurality of modes of operation may include an AC mode, or one of a plurality of DC modes. In such an aspect, the plurality of DC modes may include: a first DC mode with a first gain and a first level of sensitivity, a second DC mode with a second gain and a second level of sensitivity, wherein the second gain is greater than the first gain and wherein the second level of sensitivity is greater than the first level of sensitivity, and a third DC mode with a third gain and a third level of sensitivity, wherein the third gain is greater than the first gain and second gain and wherein the third level of sensitivity is greater than the first level of sensitivity and the second level of sensitivity. Further, in such an aspect, the first gain may be approximately 1.5+/−0.1, the first level of sensitivity may be +/−1.3V+/−0.1V, the second gain may be 6.3+/−0.1, the second level of sensitivity may be +/−325 mV+/−25 mV, the third gain may be 12.7+/−0.1, and the third level of sensitivity may be +/−160 mV+/−25 mV. In another aspect, the AC mode may use a gain of 400+/−10 and a level of sensitivity of +/−5 mV+/−1 mV.

At reference numeral 304, a plurality of input signals may be received over a plurality of input channels. In one aspect, the input signals may be measuring various values, such as, but not limited to, EEG measurements, TCD measurements, perfusion measurements, ICP measurements, invasive depth EEG measurements, blood pressure measurements, temperature measurements, heart rate measurements, SpO₂ measurements, etc.

At reference numeral 306, received input signals may be processed using the selected first mode. In one aspect, the processed data may be stored on the device. In another aspect, the processed data may be transmitted (e.g., wirelessly or through a wired or fiber optic connection) to a network for subsequent aggregation, analysis, etc. In another aspect, the processed data may be transmitted continuously, periodically, and/or upon receiving a data request.

At reference numeral 308, a selection may be received to change the device mode of operation to a second mode of the plurality of modes. In one aspect, the change may be due to a change in the stage of care in which the device is operable, for example. Each stage of care may weigh different performance parameters differently. Performance parameters may include, but are not limited to input signal sampling rate, noise in the input signals, a common mode rejection ratio (CMRR) value, cost of equipment, device battery life, device memory capacity, device size, DC offset tolerance, input bias current, bandwidth sensitive range, etc.

At reference numeral 310, the device may process the received signals using the second mode of operation. Further, the changing to the second mode of operation for processing of the received signals may be performed without signal reception interruption. In other words, the device allows for continuous signal reception across multiple stages of care.

Referring to FIG. 4, a flowchart of an example course of events 400, in accordance with aspect of the present invention in which a data acquisition device may be used is illustrated. At reference numeral 402, an event may occur for an individual, such as a seizure, heart failure, a concussion, etc. At reference numeral 404, the device may be connected to the affected individual. Further, the device may be set to operate in a first stage of care 418 (e.g., LTM monitoring, clinical EGG operation). At reference numeral 406, the affected individual may be transported to the hospital. In one aspect, the device may communicate stored data to care providers in the ambulance using a lap top computer, or the like. At reference numeral 408, the affected individual may arrive at the hospital and undergo further examination. In one aspect, the device may be changed to a second mode of operation associated with a second stage of care 420 (e.g., Emergency Room, ICU, research study). At reference numeral 410, upon an occurrence of a surgical procedure, the device may be changed to a third mode of operation associated with a third stage of care 422 (e.g., OR operations). At reference numeral 412, upon completion of the surgical procedure, the device may be changed to a fourth mode of operation associated with a fourth stage of care 424 (e.g., ICU, LTM, research study). At reference numeral 414, the individual may be released from the hospital, and the device may change to a fifth mode of operation associated with a fifth stage of care 426 (e.g., Ambulatory, sleep studies). Thereafter, the device may be removed at reference numeral 416.

The previous description is provided to enable a person skilled in the art to fully understand the full scope of the disclosure. Modifications to the various configurations disclosed herein will be readily apparent to those skilled in the art. Thus, the claims are not intended to be limited to the various aspects of the disclosure described herein, but is to be accorded the full scope consistent with the language of claims, wherein reference to an element in the singular is not intended to mean “one and only one” unless specifically so stated, but rather “one or more.” Unless specifically stated otherwise, the term “some” refers to one or more. A claim that recites at least one of a combination of elements (e.g., “at least one of A, B, or C”) refers to one or more of the recited elements (e.g., A, or B, or C, or any combination thereof). All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.”

In one or more exemplary aspects, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that may be accessed by a computer. By way of example, and not limitation, such computer-readable media may include RAM, ROM, EEPROM, CD-ROM or other optical disk storage, magnetic disk storage or other magnetic storage devices, or any other medium that may be used to carry or store desired program code in the form of instructions or data structures and that may be accessed by a computer. Also, any connection is properly termed a computer-readable medium. For example, if the software is transmitted from a website, server, or other remote source using a coaxial cable, fiber optic cable, twisted pair, digital subscriber line (DSL), or wireless technologies, such as infrared, radio, and microwave, then the coaxial cable, fiber optic cable, twisted pair, DSL, or wireless technologies, such as infrared, radio, and microwave, are included in the definition of medium. Disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

All references cited herein are hereby incorporated by reference in their entirety.

What is claimed is:

1. A data acquisition device, comprising:
two inputs coupled to an instrumentation amplifier, wherein each input provides an input signal to the instrumentation amplifier;
a mode selection module, coupled to the instrumentation amplifier, operable to select either an alternating current (AC) mode, or one of a plurality of direct current (DC) modes for processing the input signals, wherein the mode is determined based at least in part on analyzing at least one of a plurality of parameters associated with a plurality of stages of care; and
a data collection module operable to process the received signals using the selected mode.

2. The data acquisition device of claim 1, wherein the selected stage of care of the plurality of stages of care is changed to a different stage of care, and wherein the data collection module is further operable to continuously process the received signals, when the selected stage of care of the plurality of stages of care is changed to the different stage of care.

3. The data acquisition device of claim 2, wherein the mode is changed when the selected stage of care of the plurality of stages of care is changed to the different stage of care.

4. The data acquisition device of claim 1, wherein the plurality of DC modes comprise:
a first DC mode with a first gain and a first level of sensitivity;
a second DC mode with a second gain and a second level of sensitivity, wherein the second gain is greater than the first gain and wherein the second level of sensitivity is greater than the first level of sensitivity; and
a third DC mode with a third gain and a third level of sensitivity, wherein the third gain is greater than the first gain and second gain and wherein the third level of sensitivity is greater than the first level of sensitivity and the second level of sensitivity.

5. The data acquisition device of claim 4, wherein the first gain is within a range of +/−0.1 of 1.50, the first level of sensitivity is within a range of +/−0.1 mV of +/−1.3V, the second gain is within a range of +/−0.1 of 6.3, the second level of sensitivity is within a range of +/−25 mV of +/−325 mV, the third gain is within a range of +/−0.1 of 12.7, and the third level of sensitivity is within a range of +/−25 mV of +/−160 mV.

6. The data acquisition device of claim 1, wherein the AC mode comprises a gain within a range of +/−10 of 400 and a level of sensitivity within a range of +/−1 mV of +/−5 mV.

7. The data acquisition device of claim 1, wherein the plurality of parameters comprise at least two selected from a group consisting of:
input signal sampling rate;
noise in the input signals;
a common mode rejection ratio (CMRR) value;
cost of equipment;
device battery life;
device memory capacity;
device size;
DC offset tolerance;
input bias current; and
bandwidth sensitive range.

8. The data acquisition device of claim 1, wherein the plurality of stages of care comprise at least two selected from a group consisting of:
clinical electroencephalography (EEG) operation;
long term monitoring (LTM) operation;
intensive care unit (ICU) operation;
operating room (OR) operation;
sleep study operation;
research study operation; and
ambulatory operation.

9. The data acquisition device of claim 1, wherein the input signals are used to acquire at least one selected from a group consisting of:
EEG measurements;
transcranial Doppler (TCD) measurements;
perfusion measurements;
intracranial pressure (ICP) measurements;
invasive depth EEG measurements;
blood pressure measurements;
temperature measurements;
heart rate measurements;
Saturation of peripheral oxygen (SpO₂) measurements;
evoked potential measurements; and
vital sign monitoring measurements.

10. The data acquisition device of claim 1, wherein the instrumentation amplifier is powered using a dual supply, and wherein the mode selection module is powered using a single supply.

11. The data acquisition device of claim 10, further comprising:
a data storage module operable to store data associated with at least a portion of the processed signals generated by the data collection module.

12. The data acquisition device of claim 11, further comprising:
a data transmission module operable to transmit at least a portion of the stored data.

13. The data acquisition device of claim 12, wherein the data transmission module is further operable to transmit using at least one selected from a group consisting of:
wireless transmitter;
a wireline connection; and
a fiber optic coupling.

14. The data acquisition device of claim 12, wherein the data transmission module is further operable to transmit through at least one selected from a group consisting of:
continuous transmission;
periodic transmission; and
transmission in response to a received data request.

15. A method of continuously acquiring data, the method comprising:
receiving a selection of a first mode of operation from a plurality of modes, wherein the mode is determined based at least in part on analyzing at least one of a plurality of parameters associated with a plurality of stages of care;
receiving input signals from two inputs;
processing the received input signals using the selected first mode of operation to generate a first data set;
receiving a selection of a second mode of operation from the plurality of modes, wherein the second mode differs from the first mode; and
processing the received input signals using the selected second mode of operation without interruption in receiving the input signals to generate a second data set.

16. The method of claim 15, wherein the receiving a selection of the second mode further comprises:
detecting a change to a new stage of care of the plurality of stages of care; and
selecting the second mode in response to the detected change.

17. The method of claim 15, wherein the plurality of modes of operation comprise an AC mode or one of a plurality of DC modes for processing the input signals.

18. The method of claim 17, wherein the plurality of DC modes comprise:
a first DC mode with a first gain and a first level of sensitivity;
a second DC mode with a second gain and a second level of sensitivity, wherein the second gain is greater than the first gain and wherein the second level of sensitivity is greater than the first level of sensitivity; and
a third DC mode with a third gain and a third level of sensitivity, wherein the third gain is greater than the first gain and second gain and wherein the third level of sensitivity is greater than the first level of sensitivity and the second level of sensitivity.

19. The method of claim 18, wherein the first gain is within a range of +/−0.1 of 1.50, the first level of sensitivity is within a range of +/−0.1 mV of +/−1.3V, the second gain is within a range of +/−0.1 of 6.3, the second level of sensitivity is within a range of +/−25 mV of +/−325 mV, the third gain is within a range of +/−0.1 of 12.7, and the third level of sensitivity is within a range of +/−25 mV of +/−160 mV.

20. The method of claim 17, wherein the AC mode comprises a gain of 406 and a level of sensitivity of +/−5 mV.

21. The method of claim 15, wherein the plurality of parameters comprise at least two selected from a group consisting of:
input signal sampling rate;
noise in the input signals;
a common mode rejection ratio (CMRR) value;
cost of equipment;
device battery life;
device memory capacity;
device size;
DC offset tolerance;
input bias current; and
bandwidth sensitive range.

22. The method of claim 15, wherein the plurality of stages of care comprise at least two selected from a group consisting of:
clinical electroencephalography (EEG) operation;
long term monitoring (LTM) operation;
intensive care unit (ICU) operation;
operating room (OR) operation;
sleep study operation;
research study operation; and
ambulatory operation.

23. The method of claim 15, wherein the input signals are used to acquire at least one selected from a group consisting of:
EEG measurements;
transcranial Doppler (TCD) measurements;
perfusion measurements;
intracranial pressure (ICP) measurements;
invasive depth EEG measurements;
blood pressure measurements;
temperature measurements;
heart rate measurements;
Saturation of peripheral oxygen (SpO₂) measurements;
evoked potential measurements; and
vital sign monitoring measurements.

24. The method of claim 15, further comprising:
storing at least a portion of the first data set and the second data set; and
transmitting at least a portion of the stored data.

25. The method of claim 24, wherein transmitting of at least a portion of the stored data further comprises:
transmitting wirelessly, through a wireline connection, or via a fiber optic coupling.

26. The method of claim 24, wherein the transmitting further comprises:
transmitting through at least one selected from a group consisting of: continuous transmission, periodic transmission, and transmission in response to a received data request.
